(19) United States
(12) Patent Application Publication
(10) Pub. No.: US 2004/0022742 A1
(43) Pub. Date: Feb. 5, 2004

(54) Liquid deodorant composition and method for use thereof

(76) Inventors: Toshiyuki Isobe, Fukuoka-Ken (JP); Kunjo Sasada, Kumamoto-Ken (JP); Shuichi Sugihara, Tokyo-To (JP); Ryota Shinohara, Kumamoto-ken (JP)

Correspondence Address: LADAS & PARRY, 26 WEST 61ST STREET, NEW YORK, NY, 10023, US

(21) Appl. No.: 10/380,972

(22) PCT Filed: Sep. 21, 2001
(86) PCT No.: PCT/JP01/08275

(30) Foreign Application Priority Data
Sep. 21, 2000 (JP) 2000-286673

(51) Int. Cl. A61L 9/00; A61L 9/01

(52) U.S. Cl. 424/47; 424/76.1

(57) ABSTRACT

A liquid deodorant composition comprising an aqueous solution containing specific 3d transition metal halides is provided. This deodorant composition does not require high facility and operating costs, and can effectively reduce malodors, especially sulfureous and nitrogenous offensive odor substances.

### BACKGROUND OF THE INVENTION

1. Field of the Invention

The present invention relates to a liquid deodorant composition having the effect of reducing malodors emitted from sulfureous offensive odor substances (hydrogen sulfide, methyl mercaptan, methyl sulfide, methyl disulfide, etc.), nitrogenous offensive odor substances (ammonia, trimethylamine, etc.) and the like, useful for deodorizing, for example, cattle stalls, especially domestic animals and/or their excreta.

2. Background Art

In recent years, the scale of stockbreeding business has been rapidly enlarged, while housing sites have been extended to the suburbs. As a natural consequence of this trend, a lot of complaints on the management of stockbreeding are now being echoed among suburbanites, the majority of the complaints being related to malodors.

To reduce malodors in stock farms, there has usually been adopted such a method that malodorous gases emitted are removed by allowing activated carbon, zeolite or the like to adsorb the gases, or that waste water, the source of an offensive odor, is treated by an active sludge process or the like. These conventional methods, however, are not of such a type that a deodorant is sprinkled directly on domestic animals and their excreta to remove the roots of offensive odors themselves. They have therefore a limitation on treatment capability, and, in addition, require high facility and operating costs.

### SUMMARY OF THE INVENTION

We now found that it is possible to effectively reduce malodors, especially those ones emitted from sulfureous offensive odor substances (hydrogen sulfide, methyl mercaptan, methyl sulfide, methyl disulfide, etc.), nitrogenous offensive odor substances (ammonia, trimethylamine, etc.) and the like by sprinkling aqueous solutions containing specific 3d transition metal halides on the sources of the malodors (e.g., domestic animals and their excreta).

An object of the present invention is therefore to provide a liquid deodorant composition that does not require high facility and operating costs and that can effectively reduce malodors, especially sulfureous and nitrogenous offensive odor substances.

To attain the above object, the present invention provides a liquid deodorant composition comprising an aqueous solution containing specific 3d transition metal halides.

The present invention also provides a method of using a liquid deodorant composition, comprising the step of spraying the liquid deodorant composition.

### DETAILED DESCRIPTION OF THE INVENTION

A liquid deodorant composition of the present invention, and a process of using the same will be concretely described hereinafter.

Liquid Deodorant Composition

A liquid deodorant composition of the present invention comprises an aqueous solution containing specific 3d transition metal halides.

Any 3d transition metal halide can herein be used as long as it is ionizable in an aqueous solution, and a variety of halides can be used. Highly ionizable halides (e.g., chlorides and bromides) are preferred because they have an excellent deodorizing effect; and strong electrolytes, which are fully ionizable in solutions, are more preferred. The term “3d transition metals” as used herein denotes a series of metals having filled 3d shells, which are the transition metals from ₂₁Sc to ₂₉Cu also referred to as the elements of the first transition series.

According to the present invention, it is possible to effectively reduce malodors, especially those ones emitted from sulfureous offensive odor substances (hydrogen sulfide, methyl mercaptan, methyl sulfide, methyl disulfide, etc.), nitrogenous offensive odor substances (ammonia, trimethylamine, etc.) and the like by sprinkling the aqueous solution containing specific 3d transition metal halides on the sources of the malodors (e.g., domestic animals and their excreta).

Without the intention of being bound by theory, it is believed that the deodorant action of the present invention is revealed by the following mechanism. Regarding sulfureous offensive odor substances, a) H₂S combines with 3d transition metal ions to form sparingly soluble sulfides, and b) polysulfides combine with metal ions (M) to form M₂^{I}Sₙ or M^{II}Sₙ (n=2-6), or these polysulfides formed are finally decomposed even to H₂S and followed to form sparingly soluble sulfides with the 3d transition metal ions. The sulfureous offensive odor substances are thus removed. On the other hand, ammonia, a nitrogenous offensive odor substance, forms ammine complexes [M(NH₃)₆]²⁺ or [M(NH₃)₆]³⁺ together with 3d transition metal ions, whereby ammonia is removed. Trimethylamine, another nitrogenous offensive odor substance, is also considered to cause complexation of the same type. The liquid deodorant composition of the present invention has the effect of reducing not only the above-described sulfureous and nitrogenous offensive odor substances but also many other malodorous substances. For instance, it has a deodorizing effect also on lower fatty acid malodorous substances (e.g., n-butyric acid and isovaleric acid) because metal ions, especially strontium, contained in the deodorant composition form salts or complex salts together with these malodorous substances.

According to a preferred embodiment of the present invention, the 3d transition metal halides comprise iron(III) chloride, manganese(II) chloride, cobalt(II) chloride, nickel(II) chloride, or a combination thereof, more preferably, iron(III) chloride, manganese(II) chloride, and cobalt(II) chloride and/or nickel(II) chloride.

According to a preferred embodiment of the present invention, the deodorant composition further comprises alkaline metal halides and/or alkaline-earth metal halides, more preferably both alkaline metal halides and alkaline-earth metal halides. When these halides are present in the deodorant composition, it is easy to maintain the composition at a nearly neutral pH (preferably pH=6-8, more preferably pH=6.5-7.5), and the deodorizing effect of 3d transition metal ions is thus enhanced. Moreover, alkaline metal ions/alkaline-earth metal ions (M) are considered to have some deodorant action because they themselves form M₂^{I}Sₙ or M^{II}Sₙ (n=2-6) together with the polysulfides, or because these polysulfides formed are finally decomposed even to H₂S and followed to form sparingly soluble sulfide with the 3d transition metal ions. This means that the deodorizing effect can be efficiently obtained on a variety of malodorous substances.

Any alkaline metal halide can be used in the present invention. The alkaline metal halides preferably comprise sodium chloride, potassium chloride, lithium chloride, potassium bromide, or a combination thereof. More preferably, the alkaline metal halides comprise all of the above-enumerated halides.

Any alkaline-earth metal halide can be used in the present invention. The alkaline-earth metal halides preferably comprise barium chloride, calcium chloride, magnesium chloride, strontium chloride, or a combination thereof. More preferably, the alkaline-earth metal halides comprise all of the above-enumerated halides.

According to a preferred embodiment of the present invention, the liquid deodorant composition has a pH of 6 to 8, more preferably 6.5 to 7.5, most preferably 6.8 to 7.2.

The concentration of the liquid deodorant composition of the present invention may be varied depending on its use, the manner in which the composition is used, etc., and there is no particular limitation on it. According to a preferred embodiment of the present invention, the ionic strength of the deodorant composition is 3 or more. As long as the liquid deodorant composition has an ionic strength in this range, it can be presented as a concentrated liquid, which is favorable for storage, transportation, and the like. For instance, in the case where the deodorant composition is sprinkled directly on domestic animals, the following manner is available: the composition is firstly prepared as a concentrated liquid, and, upon use, diluted effectively to a desired concentration. According to another preferred embodiment of the present invention, the deodorant composition has an ionic strength of 0.5 or less. When the liquid deodorant composition has an ionic strength in this range, it can fully and efficiently show its deodorizing effect, compared when used as it is, and, moreover, does not show any adverse effect on ecosystem (e.g., domestic animals) and environment.

The liquid deodorant composition of the present invention can further comprises any kinds of optional ingredients, depending on an intended use within the scope of the purpose of the present invention. According to a preferred embodiment of the present invention, the liquid deodorant composition further comprises a surfactant in order to provide a detergent effect as well as the deodorant effect. Such a surfactant-containing liquid deodorant composition can preferably be used for stockbreeding facilities, industrial wastes treating facilities, general wastes treating facilities, wastes collecting facilities, toilets, drains, oil-based wastes treating facilities, foods treating facilities, nursing homes, and pet shops. According to a more preferred embodiment of the present invention, the liquid deodorant composition, which preferably comprises the surfactant, further comprises a germicide to provide a germicidal effect.

Production Process

A liquid deodorant composition of the present invention can be produced by any proper method selected depending on the type of substances to be used. For example, the process mentioned below may be employed to produce a deodorant composition containing chosen 3d transition metal ions, alkaline metal ions and alkaline-earth metal ions. Alkaline metal halides are placed in a first vessel, while alkaline-earth metal halides and 3d transition metal halides are placed in a second vessel. Hot water heated to a temperature of approximately 90° C. or more is poured into the first and second vessels in a volume ratio ranging from 6:4 to 8:2, followed by stirring. There after, the aqueous solution in the first vessel and that in the second vessel are mixed each other. The mixture is stirred again, and then cooled to room temperature to give a liquid deodorant composition of the present invention.

Uses

Malodorous substances are discharged from factories, industrial facilities, etc. in various manners as described in the following items i) to iii), and the liquid deodorant composition of the present invention can be favorably used in any of these cases:

i): There is no particular smokestack, and malodorous substances are discharged from the whole facility sites as in the case of cattle stalls and the like;

ii): Offensive odor substances are discharged from smokestacks or other exhaust systems as in the case of chemical plants and the like; and

iii): Waste water containing malodorous substances is discharged from industrial facilities and evaporated as in the case of dead animal processing plants and the like.

It is particularly preferable to apply the liquid deodorant composition of the invention to cattle stalls described in the above item i). One reason for this is that both sulfureous and nitrogenous offensive odor substances, which are the major objectives of deodorization in the invention, are produced in large quantities in cattle stalls. Another reason is that it is possible to make the deodorant composition of the invention directly act on domestic animals and their excreta. In addition, the deodorant composition of the invention is composed of components not harmful to men and cattle, so that it is particularly suitable for the deodorization of domestic animals and their excreta. Examples of cattle stalls to which the deodorant composition of the invention can be applied include cowhouses, pigpens, and henhouses. Further, it is also preferable to use the deodorant composition of the invention for deodorizing waste water described in the above item iii). In this case, it is possible to precipitate malodorous substances, so that the treatment of waste water can easily be carried out.

Uses of the liquid deodorant composition of the present invention are not limited to the above-described ones. The deodorant composition of the invention can be widely used for the purpose of deodorizing both household and public facilities including household toilets and communal lavatories.

Method of Using Liquid Deodorant Composition

Any one of various known methods, such as spraying or mixing, can be adopted as a method of using the liquid deodorant composition of the present invention as long as it can make the composition exist on the sources of offensive odors or in malodorous atmospheres.

According to a preferred embodiment of the present invention, the sprinkling of the liquid deodorant composition is conducted by spraying to attain efficient deodorization. The pressure to be applied to the deodorant composition upon spraying is preferably 1.5 atm or more, more preferably from 1.5 to 3.5 atm, particularly from 2.0 to 2.5 atm. By spraying the deodorant composition at a pressure in the above range, a further improved deodorizing effect can be obtained. According to a preferred embodiment of the present invention, the above method further comprises, before the step of spraying, the step of diluting the liquid deodorant composition to make its ionic strength 0.5 or less.

Thus, the deodorant composition of the present invention can show an excellent deodorizing effect even when it is used by such a simple method that the composition is directly sprinkled on an object to be deodorized. The deodorant composition of the invention is therefore advantageous in that it can minimize the facility and operating costs.

### EXAMPLES

The present invention will now be explained more clearly by referring to the following examples. However, these examples are not intended to limit or restrict the scope of the invention in any way.

### Example 1

This example shows a case where a liquid deodorant composition of the present invention was used for deodorizing the interior of a windowless pigpen (the number of pigs accommodated: approximately 4,000 head, the site area: 2,800 m²).

(1) Preparation of Liquid Deodorant Composition

The following compounds were firstly prepared: (a) 8 g of cobalt chloride and 7 g of manganese chloride as the 3d transition metal halides; (b) 60 g of sodium chloride, 20 g of potassium chloride, 5 g of lithium chloride and 10 g of potassium bromide as the alkaline metal halides; and (c) 15 g of barium chloride, 15 g of calcium chloride, 20 g of magnesium chloride and 10 g of strontium chloride as the alkaline-earth metal halides.

Next, the above-described alkaline metal halides (b) were placed in vessel I, while the 3d transition metal halides (a) and the alkaline-earth metal halides (c) were placed in another vessel II. Hot water at a temperature of approximately 90° C. or more was poured into the vessel I in an amount of 700 ml, and to the vessel II in an amount of 300 ml, followed by rapid stirring. The contents of the vessels I and II were simultaneously transferred to still another vessel III, and the mixture was stirred. The mixture was then air-cooled at normal temperature to give a liquid deodorant composition of the present invention. The liquid deodorant composition obtained was found to have a pH ranging from 6.8 to 7.2, and an ionic strength of 3.29.

(2) Deodorization Test

The initial concentrations of malodorous substances present in the pigpen were measured in accordance with Akushu Boshi-Ho (or “Japanese Foul Smell Control Law”) (Recommendation No. 9, Attached Table 2, 1972). Specifically, the concentrations of i) ammonia and trimethylamine, nitrogenous offensive odor substances, ii) hydrogen sulfide and methyl mercaptan, sulfureous offensive odor substances, and iii) n-butyric acid and isovaleric acid, lower fatty acid substances, were respectively measured in the manners described below. The results are shown in Table 1.

a) Measurement of Concentration of Ammonia:

A boric acid solution was allowed to absorb a sample to collect ammonia. A color-developing liquid was added to this ammonia-containing solution, and the absorbance was determined at about 640 nm by the use of a spectrophotometer. The concentration of ammonia was calculated from the absorbance determined.

b) Measurement of Concentration of Trimethylamine:

A sulfuric acid solution was allowed to absorb a sample to collect trimethylamine. This trimethylamine-containing solution was placed in a decomposition bottle containing a potassium hydroxide solution. Trimethylamine generated from the decomposition bottle was introduced to a sample-concentrating tube cooled by liquid oxygen, and concentrated at a low temperature. The sample-concentrating tube was then connected to a gas chromatograph (GC) equipped with a hydrogen flame ionization detector, and heated to transfer the trimethylamine to the GC column for analysis.

c) Measurement of Concentrations of Hydrogen Sulfide and Methyl Mercaptan:

A sample collected in a sample-collecting bag was passed through a sample-concentrating tube cooled by liquid oxygen, thereby concentrating hydrogen sulfide and methyl mercaptan, sulfur compounds, at a low temperature. The sample-concentrating tube was then connected to a gas chromatograph (GC) equipped with a flame photo-detector (FPD), and heated to transfer the sulfur compounds to the GC column for analysis.

d) Measurement of Concentrations of n-Butyric Acid and Isovaleric Acid:

A sample was passed, at normal temperature, through a sample-collecting tube filled with glass beads coated with strontium hydroxide, thereby collecting n-butyric acid and isovaleric acid. The sample-collecting tube was then connected to a gas chromatograph (GC) equipped with a hydrogen flame ionization detector, and formic acid was injected. Thereafter, the sample-collecting tube was heated to transfer the n-butyric acid and isovaleric acid to the GC column for analysis.

Next, the deodorization of the pigpen was conducted by the use of the liquid deodorant composition of the present invention. 60 liters of the liquid deodorant composition was firstly diluted with water to 2,000 liters, thereby obtaining a dilute solution to be sprinkled for one day; this quantity of the dilute solution was equivalent to 0.5 liters per day per 50 kg of pig. The ionic strength of the dilute solution was found to be 0.1. This dilute solution was divided into 12, and regularly sprinkled on the interior of the whole pigpen, where the dilute solution was each time jetted over a period of 3 minutes from a sprinkler laid on the ceiling of the pigpen. This sprinkling operation was conducted for 60 consecutive days. The concentrations of the malodorous substances present in the pigpen were then measured in accordance with the above-described procedures from a) to d).

3) Test Results

The results of the deodorization test described in the above item (2) are shown in Table 1 below.

TABLE 1

| | | Concentrations after |
|---|---|---|
| | Initial | Sprinkling for 60 |
| Malodorous Substances | Concentrations (ppm) | Days (ppm) |
| (Nitrogenous Offensive | | |
| Odor Substances) | | |
| Ammonia | 8.5 | 2.5 |
| | | non-detectable (less |
| Trimethylamine | 0.002 | than 0.005 ppm) |
| (Sulfureous Offensive | | |
| Odor Substances) | | |
| Hydrogen Sulfide | 0.016 | 0.03 |
| Methyl mercaptan | 0.42 | 0.003 |
| (Lower Fatty Acid | | |
| Substances) | | |
| n-butyric Acid | 0.25 | 0.13 |
| Isovaleric Acid | 0.065 | 0.012 |

The data shown in Table 1 demonstrate that all of the nitrogenous offensive odor substances, sulfureous offensive odor substances and lower fatty acid substances were greatly reduced by the liquid deodorant composition. In other words, the liquid deodorant composition showed an excellent deodorizing effect.

When discussing the data shown in Table 1, attention should be paid on the following points:

i) the pigs grew day by day, so that the amount of their excreta per day also increased with the passage of time;

ii) the deodorant composition was sprinkled (not sprayed) from day 1 to day 24, and then sprayed at a water pressure of 2.2 atm from day 25 to day 60; and

iii) although the spraying of the deodorant was completed on day 60, the concentrations of the malodorous substances were actually measured on day 78 due to various reasons; during this blank period, the animals voided excreta, so that the quantities of the malodorous substances increased.

If the above points are taken into consideration, it is believed that the practical degree of deodorization is much higher than the degree known from the data shown in Table 1.

### Example 2

This example shows a case where a liquid deodorant composition of the present invention was used for deodorizing waste water discharged from the dehydration treatment of sludge that had been produced during a paper making process.

(1) Preparation of Liquid Deodorant Composition

A liquid deodorant composition was prepared in the same manner as in Example 1.

(2) Deodorization Test

1.1 ml of the liquid deodorant composition was added to 110 ml of the waste water, and the mixture was stored in an airtight container at room temperature. After 1 hour, the mixture was passed through a 0.45-μm filter, and the filtrate was used as a sample in the subsequent step.

The concentrations of hydrogen sulfide, methyl mercaptan, methyl sulfide and methyl disulfide in the sample were measured in accordance with Akushu Boshi-Ho (or “Japanese Foul Smell Control Law”) (Recommendation No. 9, Attached Table 2, 1972). Specifically, the sample placed in a sample-collecting bag was passed through a sample-concentrating tube cooled by liquid oxygen, thereby concentrating the sulfur compounds at a low temperature. The sample-concentrating tube was then connected to a gas chromatograph (GC) equipped with a flame photo-detector (FPD), and heated to transfer the sulfur compounds to the GC column for analysis. The results of the analysis are shown in Table 2.

### Comparative Example 1

The procedure of Example 2 was repeated to prepare a sample and to measure the concentrations of the malodorous substances in the sample, provided that the liquid deodorant composition was not used. The results are shown in Table 2.

### Example 3

The procedure of Example 2 was repeated to prepare a sample and to measure the concentrations of the malodorous substances in the sample, provided that 10 ml of the liquid deodorant composition was added to 100 ml of the waste water. The results are shown in Table 2.

### Comparative Example 2

The procedure of Example 3 was repeated to prepare a sample and to measure the concentrations of the malodorous substances in the sample, provided that the liquid deodorant composition was not used. The results are shown in Table 2.

TABLE 2

| | Hydrogen | Methyl | Methyl | Methyl |
|---|---|---|---|---|
| | Sulfide | Mercaptan | Sulfide | Disulfide |
| Original Waste | 12.8 | 0.665 | 0.04 | ND |
| Water | | | | |
| Example 2 | 1.04 | 0.019 | ND | ND |
| Comp. Ex. 1 | 3.84 | 0.103 | 0.01 | ND |
| Example 3 | ND | ND | ND | ND |
| Comp. Ex. 2 | 2.29 | 0.065 | 0.01 | ND |
| Lower Limit of | 0.001 | 0.001 | 0.01 | 0.02 |
| Determination | | | | |

Unit: mg/l
ND: less than the lower limit of determination

### Example 4

This example shows a case where the liquid deodorant composition of the present invention was used for the deodorization of an ammonia solution and the maintenance of the deodorizing effect was examined.

(1) Preparation of Liquid Deodorant Composition

A liquid deodorant composition was prepared in the same manner as in Example 1.

(2) Deodorization Test

0.6 ml of the liquid deodorant composition was added to 0.1 ml of an ammonia solution having the ammonia concentration of 10,000 ppm, and the mixture thus obtained was placed in a chamber. After 20 minutes and 30 minutes from the mixing, ammonia concentration of the mixture was measured by a detector tube. During the measure, fans positioned in the chamber were operating in order to make ammonia concentration even within the chamber. The result thus obtained is shown in Table 3.

### Example 5

The procedure of Example 4 was repeated to prepare a sample and to measure the concentrations of the malodorous substances in the sample, provided that 23 g of cobalt chloride and 17 g of manganese chloride were employed to increase the amount of 3d transition metal halides. The result thus obtained is shown in Table 3.

### Comparative Example 3

The procedure of Example 4 was repeated to prepare a sample and to measure the concentrations of the malodorous substances in the sample, provided that 0.6 ml of water was employed without using 0.6 ml of the liquid deodorant. The result thus obtained is shown in Table 3.

TABLE 3

| | After 20 Minutes | After 30 Minutes |
|---|---|---|
| Example 4 | 5 ppm | 5 ppm |
| Example 5 | 2 ppm | 2 ppm |
| Comparative Example 3 | 22 ppm | 22 ppm |

The data shown in Table 3 demonstrate that, in a case where the liquid deodorant composition of the present invention was used (Examples 4 and 5), significantly improved ammonia deodorant effect was attained, compared to a case where water was used (Comparative Example 3). In view of the comparison between Example 4 and Example 5, it can be seen that the increased amount of 3d transition metal halides further improve the deodorant effect. It can also be seen that the deodorant effect is maintained for a long period of time. It is believed that these results mean that ammonia once absorbed or fixed to the liquid deodorant composition would not be released again.

1. A liquid deodorant composition comprising an aqueous solution containing specific 3d transition metal halides.

2. The liquid deodorant composition according to claim 1, wherein the 3d transition metal halides comprise at least more than one halide selected from the group consisting of iron (III) chloride, manganese (II) chloride, cobalt (II) chloride and nickel (II) chloride.

3. The liquid deodorant composition according to claim 2, wherein the 3d transition metal halides comprise manganese (II) chloride, and cobalt (II) chloride and/or nickel (II) chloride.

4. The liquid deodorant composition according to any of claims 1 to 3, further comprising alkaline metal halides and/or alkaline-earth metal halides.

5. The liquid deodorant composition according to claim 4, comprising both alkaline metal halides and alkaline-earth metal halides.

6. The liquid deodorant composition according to claim 4 or 5, wherein the alkaline metal halides comprise sodium chloride, potassium chloride, lithium chloride and potassium bromide.

7. The liquid deodorant composition according to any of claims 4 to 6, wherein the alkaline-earth metal halides comprise barium chloride, calcium chloride, magnesium chloride and strontium chloride.

8. The liquid deodorant composition according to any of claims 1 to 7, having a pH between 6 and 8.

9. The liquid deodorant composition according to any of claims 1 to 8, having an ionic strength of 3 or more.

10. The liquid deodorant composition according to any of claims 1 to 8, having an ionic strength of 0.5 or less.

11. The liquid deodorant composition according to any of claims 1 to 10, suitable for use in the deodorization of cattle stalls.

12. The liquid deodorant composition according to any of claims 1 to 11, suitable for use in the deodorization of domestic animals and/or their excreta.

13. The liquid deodorant composition according to any of claims 1 to 12, suitable for use in the deodorization of waste water.

14. The liquid deodorant composition according to any of claims 1 to 13, further comprising a surfactant and/or a germicide.

15. A method of using a liquid deodorant composition, comprising the step of spraying a liquid deodorant composition set forth in any of claims 1 to 14.

16. The method according to claim 14 or 15, further comprising, before the step of spraying, the step of diluting the liquid deodorant composition to make its ionic strength 0.5 or less.
